(19) United States
(12) Patent Application Publication
(10) Pub. No.: US 2021/0378749 A1
(43) Pub. Date: Dec. 9, 2021

(54) METHOD AND DEVICE FOR MONITORING IMAGES BY MEANS OF AN X-RAY DEVICE DURING A SURGICAL PROCEDURE

(71) Applicant: Ziehm Imaging GmbH, Nuremberg (DE)

(72) Inventors: Thomas König, Nuremberg (DE); Klaus Hörndler, Nuremberg (DE); Eva-Maria Ilg, Nuremberg (DE); Christof Fleischmann, Möhrendorf (DE); Andreas Horn, Stein (DE)

(21) Appl. No.: 17/313,256

(22) Filed: May 6, 2021

(30) Foreign Application Priority Data
Jun. 4, 2020 (DE) 10 2020 003 366.0

(51) Int. Cl. A61B 34/10; A61B 90/00

(52) U.S. Cl. CPC A61B 34/10; A61B 2034/107; A61B 2090/3764; A61B 90/37

(57) ABSTRACT

The present technology is the field of intraoperative imaging, wherein a planning trajectory, for example a planned drilling channel, can be displayed in a 2D X-ray image. This planning trajectory can be plotted by the surgeon in a provided 3D image data set and then displayed in the 2D X-ray image by determining the position in space via a projection geometry into an arbitrary position and orientation of a C-arm X-ray apparatus during 2D imaging.

### INCORPORATION BY REFERENCE TO ANY PRIORITY APPLICATIONS

Any and all applications for which a foreign or domestic priority claim is identified in the Application Data Sheet as filed with the present application are hereby incorporated by reference under 37 CFR 1.57.

### BACKGROUND

### Field

The present disclosure generally relates to the field of intraoperative imaging, and more specifically to display of a planning trajectory in a 2D X-ray image.

### Description of the Related Art

Significant challenges facing orthopedics and trauma surgery include the exact repositioning of dislocated bone fragments and the placement of foreign objects, such as screws, Kirschner wires and implants, as well as the correct placement of the necessary instruments. Improper positioning of such foreign objects in an examination region can lead to far-reaching health consequences, for example, posttraumatic joint stiffness, which could require further surgery. These procedures, referred to as revision surgery, mean an additional burden on the patient, as well as additional costs for the hospital where the procedures are performed.

The conventional procedure used in operating rooms to check the position of foreign objects involves the use of a C-arm X-ray apparatus. 2D imaging is the leading method here for showing the physician the current position of foreign objects.

2D imaging has proven to be disadvantageous in that it is a method of imaging in which the depth information is lost. The user therefore has to record X-ray images from a large number of positions in order to check the position of foreign objects and thus has no possibility of obtaining the position of the introduced foreign object displayed in a full-fledged 3D representation. In order to be able to assess the position of the foreign object, an attempt has therefore been made, in particular, to move the C-arm X-ray apparatus into defined positions and thus obtain an assessment from different viewing directions. This procedure requires an enormous amount of time and is associated with a large number of recordings.

The introduction of C-arms, which make it possible to create 3D volumes intraoperatively, was an improvement in quality during an operative procedure. With the aid of such C-arms, the position of the introduced foreign objects can be better verified. A disadvantage of this procedure is that only one snapshot of the position of the implant can be produced with a 3D image data set. Thus, it is not possible to track the introduction of foreign objects continuously with the aid of 3D imaging, unless a large number of 3D data sets are recorded, which means an enormous radiation exposure for the patient.

However, the use of navigation systems has made it possible to detect the position of the instruments, the patient and the geometries of the imaging in order, for example, to superimpose the 2D X-ray images on the position of the instrument and, if applicable, on a screw connected to the instrument. There are now a large number of navigation systems which utilize the 2D/3D image data in order to provide the user with assistance for the introduction of foreign objects. These systems are very complex to operate and cost-intensive to acquire, however.

Document DE 10 2010 027 692 A1 discloses a method for image monitoring during the implantation of a cochlear implant, in which a fusion image is generated from a 3D planning data set and a 2D radioscopic image. The document discloses only a method specifically for application to the implantation of a cochlear implant. Furthermore, a fusion image is determined for each individual image of a continuous recording. In a fusion image, recorded image contents of at least two images are combined and displayed together.

Document DE10 2012 215 001 A1 discloses a method for 2D-3D registration using instruments introduced into a patient for registration.

### SUMMARY

A problem addressed by the present technology is that of providing an improved method for determining a projection geometry between a three-dimensional image data set and a two-dimensional X-ray image for a better guided implantation. The problem addressed by the present technology may be solved by a method and/or a device with the features specified in the claims of the present application.

In a first aspect of the present technology, a method for monitoring images by means of an X-ray apparatus during a surgical procedure by means of 3D-2D registration using at least one foreign object in an examination region is described. The method includes providing a 3D image data set and displaying at least one layer generated from the 3D image data set on a display device; inputting a planning trajectory into at least one generated layer of the 3D image data set; recording a 2D X-ray image of an examination region by means of the X-ray apparatus, wherein the examination region contains the at least one foreign object; identifying the at least one foreign object in the 2D X-ray image that is not contained in the 3D image data set; determining an optimum projection geometry using a measure of similarity between the 3D image data set and the 2D X-ray image, wherein the at least one identified foreign object is masked; and displaying the planning trajectory in the 2D X-ray image on the display device by using the optimum projection geometry.

In some embodiments, the 2D X-ray image is a live image X-ray image recording. In some embodiments, the determination of the optimum projection geometry takes place by using an iterative and/or parallel optimization method. In some embodiments, the projection geometry is determined on a fixed grid by using a parallel method on a multiprocessor architecture. In some embodiments, the optimum projection geometry must satisfy a configurable threshold value of the similarity measure. In some embodiments, a subset of available geometric degrees of freedom is used to determine the projection geometry. In some embodiments, the planning trajectory is represented in a second display plane different from a first display plane used to display the at least one layer, and an intersection point of the planning trajectory is displayed in a third display plane. In some embodiments, when a plurality of planning trajectories are represented, they are identified differently from one another and/or individual planning trajectories are masked off. In some embodiments, movements of the X-ray apparatus and/or of an operating table are detected and included in the determination of the optimum projection geometry. In some embodiments, positions to be approached which facilitate an assessment of an intermediate operation result are determined by a criterion based on the planning trajectories. In some embodiments, the method further comprises, before recording the 2D X-ray image, calculating a virtual forward projection from the 3D image data set. In some embodiments, the method further comprises, after successful determination of an optimum projection geometry, superimposing the forward projection of the 3D image data set with the 2D X-ray image. In some embodiments, a new determination of the optimum projection geometry is triggered by operating a hand or foot switch, by changing an X-ray geometry, or by comparing a live image recording to the 2D X-ray image, wherein a new determination is triggered in the event of an excessive difference. In some embodiments, the 2D X-ray image is recorded before the input of the planning trajectory and/or registration is determined before the input of the planning trajectory. In some embodiments, the display of the planning trajectory is no longer updated, or is hidden, if no projection geometry is generated which changes or improves the similarity value the previous projection geometry by a fixed relative or absolute value.

In a second aspect, a device for recording image data sets of X-ray images, in particular a C-arm X-ray apparatus, is configured to carry out any of the methods described above. The device comprises a memory unit in which a recorded 3D image data set of X-rays is stored; a reconstruction unit in which the 3D image data set is reconstructed from X-rays to form a 3D volume; a control unit, said control unit being configured to permit determination of an optimum projection geometry between a forward projection of the 3D image data set and a recorded 2D X-ray image; an image processing unit for generating a 3D view of the 3D X-ray image data set having variable 3D views and for defining sectional planes for sectional plane image representations; and a GUI having an image output unit and an input unit for the image processing unit for inputting and changing the sectional planes and planning trajectories.

In a third aspect, a computer program product has a computer program which can be loaded directly into a memory unit of a control unit for a conical beam computer tomograph, in particular a C-arm X-ray device, with program sections that cause the conical beam computer tomograph to perform any of the methods described above when the computer program is executed in the control unit of the conical beam computer tomograph.

In a fourth aspect, a computer-readable medium has stored thereon program sections which can be read in and executed by a computer unit in order to perform any of the methods described above when the program sections are executed by the computer unit.

### BRIEF DESCRIPTION OF THE DRAWINGS

FIG. 1 shows one possible embodiment of a method according to the present technology.

FIG. 2 shows an embodiment of a determination of an optimum projection geometry.

### DETAILED DESCRIPTION

Methods according to the present technology for image monitoring by means of an X-ray machine during a surgical procedure by means of a 3D-2D registration, using at least one foreign object in an examination region, may include providing a 3D image data set and displaying at least one layer generated from the 3D image data set on a display device, inputting a planning trajectory into at least one generated layer of the 3D image data set, recording a 2D X-ray image of an examination region by means of the X-ray apparatus, wherein the examination region contains the at least one foreign object, identifying the at least one foreign object in the 2D X-ray image, which is not contained in the 3D image data set, determining an optimum projection geometry using a measure of similarity between the 3D image data set and the 2D X-ray image, the at least one identified foreign object is masked out, and displaying the planning trajectory in the 2D X-ray image by using the optimal projection geometry on the display device.

The 3D image data set contains anatomical structures of an examination region, these anatomical structures possibly comprising bones and/or blood vessels. The 3D image data set is preferably recorded intra-operatively, for example by using an intra-operative computed tomograph or a C-arm X-ray apparatus. If the method is carried out with a C-arm X-ray apparatus, the position of the C-arm X-ray apparatus can be no longer changed after the recording of the 3D image data set. It is also possible to produce the 3D image data set preoperatively by means of a computer tomograph, a magnetic resonance tomograph or a 3D capable C-arm X-ray apparatus. It is also possible to import the 3D image data set into an internal memory unit, for example an internal image data memory, or an external storage unit such as a USB stick, an external hard disk, or an online memory to which the X-ray machine implementing the method has access.

The 3D image data set can be displayed in the form of layers which are taken from the 3D image data set. These layers can be represented in the form of a multiplanar reformation (MPR), for example axially, sagittally, coronally, or layers with any desired orientation. Furthermore, the 3D image data set can be displayed in the form of a three-dimensional display, for example in the form of a semi-transparent volume display. This three-dimensional representation is preferably a supplement to the representation of the layers and contains a clearer representation for the method according to the present technology that is to be carried out.

Subsequently, a planning trajectory can be input into the representation of the 3D image data set, wherein an input can mean entering or plotting the planning trajectory by means of a computer mouse, a keyboard, a trackpad, a joystick or an electronic pen. Alternatively, the planning trajectory can be entered directly with a finger on the display device, provided that it is a touch-sensitive display device. According to the present technology, a planning trajectory may be linear or non-linear, where a linear planning trajectory may be, for example, a planned drilling channel. As already mentioned, the 3D image data set can be displayed in the form of a three-dimensional representation (volume representation) and/or in the form of layer representations, for example in the form of layer and/or projection images. After the planning trajectory has been input, it can be moved, rotated, extended, or shortened. A user-defined adaptation of the display can preferably be carried out before the input of the planning trajectory. Adaptation of the display can be advantageous, since a planning trajectory can then be plotted in a clearer display.

In some embodiments of the present technology, a 2D X-ray image can be recorded with a recording geometry before or after the input of a planning trajectory, it also being possible for the 2D X-ray image to be a live image recording from a sequence of live image X-ray images.

In some embodiments of the present technology, foreign objects present in the examination region may include screws, Kirschner wires, implants, clamps, hoses, instruments, scissors, scalpels, or combinations thereof. According to the present technology, extraneous anatomical structures located in the examination region of the 2D X-ray image can also be identified as foreign objects. For a better fixation of the examination area, the hands of the surgeon can also be recorded, for example.

After recording the 2D X-ray image that contains the at least one foreign object in the examination region, the foreign object can be identified as such, unless it is already present in the 3D image data set. The at least one foreign object can be identified by various methods which analyze, combine and evaluate the image contents of the recorded 2D X-ray image and/or the 3D image data set on the basis of various criteria or properties, for example, metal detection, intensity, texture, the calculation of a structure tensor including calculation and evaluation of the associated eigenvalues, as well as machine learning. Alternatively, the introduced foreign object and the instruments used therefor, if they are still present in the examination region, can already be identified from the knowledge of the planning trajectory in the vicinity of which the introduced foreign object and the instruments used therefor are to be expected.

An optimum projection geometry can be determined by using a measure of similarity between the 3D image data set and the 2D X-ray image, the at least one identified foreign object, which is not contained in the 3D image data set, being masked in the 2D X-ray image. The masking can be a marking out or omission or extraction of the at least one foreign object or an image region containing the foreign object during the calculation of the similarity measure. Furthermore, the optimum projection geometry can also be determined prior to the input of the planning trajectory, but in this case the 2D X-ray image must also be recorded prior to the input of the planning trajectory.

The optimum projection geometry to be determined can be ascertained by mathematical optimization of a quantitative similarity measure which contains the quality of the congruence of a two-dimensional forward projection produced from the 3D image data set and the 2D X-ray image from the examination region. The degree of similarity is optimized by varying the projection geometry under which the forward projections are calculated. Such a projection geometry can include the position of the X-ray source as well as the position and orientation of the X-ray detector. According to the present technology, each detected at least one foreign object introduced after the 3D recording has been carried out may not be included in the calculation of the degree of similarity during the optimization, and therefore this at least one foreign object does not impair the value of the degree of similarity, or does so only in a negligible manner. If no foreign object is identified or if no foreign object is present in the examination region, no image region is excluded from the calculation of the degree of similarity. If, on the other hand, the at least one foreign object is already present in the 3D image data set, it constitutes a distinctive feature for determining the optimum projection geometry and is preferably not excluded from the calculation of the similarity measure.

The optimum projection geometry can be determined by means of an iterative and/or parallel optimization method, which in particular originates from the group of non-convex optimization methods, for example simulated annealing methods or so-called genetic optimization. The optimum projection geometry may be determined using a control unit. The optimum projection geometry is preferably determined by means of a massively parallel method on a multiprocessor architecture, for example by means of a graphics processing unit (GPU). Advantageously, a significant time saving when determining the optimum projection geometry can be achieved by means of a multiprocessor architecture due to the parallel calculation made possible by the multiprocessor architecture. In iterative methods, a predefined motion grid (search space) comprising arbitrary and mutually different combinations of rotations and translations can be used. The motion grid can initially operate with a coarse resolution until a first optimum of similarity is found. The coarse resolution of the motion grid includes a significant change for each translational and/or rotational motion step. Furthermore, a higher-resolution motion grid can be used to scan the environment and a number of previously determined local similarity optima, wherein a higher-resolution motion grid has a smaller change in each translational and/or rotational motion step than the coarse resolution of the original motion grid. Alternatively, iterative optimization can also be carried out around such a provisional optimum, for example by means of a convex or non-convex optimization method.

All geometric degrees of freedom can be used to determine the projection geometry, e.g., the projection geometry can be varied by the optimization with inclusion of up to three translations and three rotations. In order to reduce the dimensionality of the motion grid and thus accelerate the calculation, however, the degrees of freedom can be restricted for determining the optimum projection geometry in real time. For example, only translations and rotations of the X-ray projection in the image plane of the X-ray projection can be taken into account. The optimum projection geometry can also be determined initially with a high or full number of geometric degrees of freedom, while further updates of the projection geometry can be carried out based on a reduced number of degrees of freedom.

According to the present technology, the planning trajectory can be displayed, after determining the optimum projection geometry, in the 2D X-ray image in a display plane in such a way that a geometric representation of the planning trajectory is projected forward onto the 2D X-ray image using the optimum projection geometry.

In embodiments of the present technology, the masking of the image regions which contain the identified foreign objects may result in a case where the remaining image regions are not sufficient to determine an optimum projection geometry, thus not satisfying a configured threshold value of the similarity measure. Such a threshold value criterion can be defined in a program such as an organ program. If this case occurs, the system can output information that too many identified foreign objects are present in the 2D X-ray image and request the user to remove identified foreign objects, preferably instruments, from the examination region. Alternatively, the system can also request the production of a new 3D image, which contains foreign objects such as screws that have been permanently introduced in the meantime and will no longer be masked out when calculating the similarity measure. Recording the new 3D image data set can be necessary especially if the anatomy of the examination region has been changed during a surgical procedure in such a way that a sufficient similarity to the previous 3D image data set can no longer be ensured.

In alternative embodiments, the present technology can represent the planning trajectory in a plurality of display planes, preferably in a second display plane which is different from the first, for example in a plane perpendicular to the first plane, and in a third display plane in which the intersection point of the planning trajectory is represented.

In alternative embodiments, more than one planning trajectory can be input and displayed on the display, wherein there is the possibility of again masking out all or selected, e.g., isolated, planning trajectories. Completely and partially masking the planning trajectories is advantageous, owing to the improved clarity of the planning trajectories shown on the display. It is also possible to characterize the different planning trajectories differently from one another, with different colors for example, or by means of different graphical representations such as dotted lines, dashed lines, or combinations of these representation variants. The advantage in these embodiments can be better clarified if there are a large number of entered planning trajectories.

In alternative embodiments, a variety of movements of an X-ray apparatus such as a C-arm X-ray apparatus or an operating table can be detected. Such movements can be determined, for example, by means of encoders (position and angle transmitters) on the corresponding adjustment axes. These detected movements can be included in the determination of the projection geometry in such a way that they serve as an initialization of the optimization or the motion pattern thereof in order to accelerate convergence, i.e. attainment of a sufficiently good similarity measure. Furthermore, this can reduce the probability of incorrect registrations in which the determined optimum projection geometry does not sufficiently approximate the actual projection geometry.

In alternative embodiments of the present technology, positions to be approached can be calculated on the basis of the planning trajectories, which positions facilitate an assessment of the intermediate operating result and can be determined on the basis of a criterion, in particular an optimization criterion. This optimization criterion is preferably formulated so as to align the X-ray apparatus in such a way that the planning trajectory is, for example, parallel or perpendicular to the image plane of the planning trajectory, thus avoiding a collision between the X-ray apparatus and the surroundings. For the adjustment of the position, the X-ray apparatus preferably has all adjustment axes at its disposal, for example those for adjustment of the orbital angle and/or the angulation angle, for geometric enlargement of the examination region, for height adjustment, for adjustment of the horizontal pivot plane of the detector, as well as the possibilities for adjusting the operating table.

In alternative embodiments of the present technology, it is possible to carry out the calculation of a virtual forward projection from the 3D image data set before recording the 2D X-ray image. This calculation can be carried out taking into account (including) the values of the different encoders of the corresponding adjustment axes of the X-ray apparatus, for example a C-arm X-ray apparatus. An advantage of these embodiments may be that the user can receive a virtual preview of the X-ray image to be expected, including the forward-projected planning trajectory. This can facilitate the surgical procedure and the positioning of the C-arm X-ray apparatus.

In a further advantageous configuration, the present technology can, after successful registration, produce a forward projection of the 3D image data set under the optimum projection geometry and superimpose it with the 2D X-ray image. In this case, for example, only the bones contained in the 3D image data set can be projected forward, with or without a planning trajectory, in order to allow the operator to assess how well the forward projection has been brought into alignment with the structures contained in the 2D X-ray image. The advantage of this configuration can be that the operator himself notices, by using a threshold value criterion for example, an incorrect registration that was not recognized by the system. In such a case, the procedure can then be carried out in a conventional manner, which prevents, for example, an implant from being inserted at the wrong location due to incorrect registration.

The present technology does not require a permanent recalculation of the optimum projection geometry to be determined. A re-determination of the projection geometry can be advantageous particularly if special events occur, for example a realignment of the C-arm or the elapse of a predetermined period of time.

In alternative embodiments, there is the possibility of triggering, for example by means of a hand or foot switch, the determination of a projection geometry when recording a 2D X-ray image. In this case, it is possible for the system to retain the previous display until the new projection geometry and a new display are determined.

Alternatively, the present technology can trigger the determination of a new projection geometry if the imaging geometry of the X-ray apparatus has changed and the system has determined this, for example by reading out encoders on corresponding adjustment axes of a C-arm X-ray apparatus or due to a change of the position of the X-ray apparatus, for example because a brake has been released. In such embodiments of the present technology, it can be advantageous to hide the previous display of the planning trajectory in such a case and to restore a display on a display device after the new optimum projection geometry has been determined. For small movements, which can be tracked by evaluating the encoder positions in the display of the planning trajectory, this is not necessary.

Alternatively, there is the possibility of triggering a recalculation of an optimum projection geometry by comparing the current 2D X-ray image to the 2D X-ray image that was previously used for determining the currently valid optimum projection geometry. If the difference between the 2D X-ray image used for the last determination of the optimum projection geometry and the current 2D X-ray image is too great, a recalculation is initiated. This case can occur especially if there is an excessively large change in the image content in the current 2D X-ray image, such as a change in the patient orientation or position. In these embodiments, it is advisable to calculate on a module close to the detector, for example by means of a real-time embedded processor. Such a calculation may in turn use a similarity measure to compare the two 2D X-ray projections. This similarity measure is not used in an optimization, but only serves as a trigger for a recalculating the projection geometry in the event of insufficient similarity, in which case it is important to mask out the at least one identified foreign object when calculating the similarity measure. The similarity measure therefore does not necessarily have to, but can, correspond to that which was used during optimization.

The embodiments of the convergence or termination criterion of the method for determining the projection geometry can be adjusted by using different criteria.

The optimization methods of the present technology can be regarded as converged if a fixed number of steps, iterations or grid refinements has been exceeded. These cases are stored as convergence criteria for the optimization process in a program, for example an organ program.

Alternatively, it is possible for the display of the planning trajectory to no longer be updated, or be hidden, if the method does not generate a projection geometry which changes or improves the similarity between the forward projection and the 2D X-ray image of the previous projection geometry by a fixed relative or absolute value. This relative or absolute value can likewise be defined in a program such as an organ program.

The present technology further comprises a device, in particular a C-arm X-ray apparatus, for example, a mobile C-arm X-ray apparatus, which produces images of image data sets from X-ray recordings. The device includes a memory unit, a reconstruction unit, a control unit, an image processing unit, and a GUI. A recorded 3D image data set of X-rays is stored in the memory unit. The reconstruction unit can reconstruct the 3D volume from the received image data set. Furthermore, the completely reconstructed 3D volume can merely be received and stored in the memory unit. In that case, the reconstruction unit may be implemented outside of the computer, but in the overall system. The control unit makes it possible to determine an optimum projection geometry between a forward projection of the 3D image data set and a recorded 2D X-ray image. An image processing unit generates a 3D view of the 3D image data set with variable 3D views. Sectional planes for a sectional plane representation can also be defined by means of the image processing unit. The device can additionally contain a GUI having an image output unit, preferably with a display device, and an input unit with which sectional planes and planning trajectories are input and changed.

A largely software-based implementation of the method has the advantage that even previously used methods for foreign object recognition for image recording systems can be retrofitted in a simple manner by a software update in order to operate according to the present technology. In this respect, the object is also achieved by a corresponding computer program product having a computer program which can be loaded directly into a memory device of an image recording system, for example a conical beam computer tomograph, having program sections in order to execute the steps of the methods according to the present technology when the computer program is executed in the control device. In addition to the computer program, such a computer program product may optionally comprise additional components such as documentation and/or additional components, including hardware components for using the software.

A computer-readable medium, for example a memory stick, a hard disk or another portable or permanently installed data carrier, on which the program sections of the computer program which can be read in and executed by a computer unit of the control device are stored, can be used for transport to the control device and/or for storage on or in the control device. A connection to a hospital information system connected to a network, to a radiology information system or to a global network, in which systems are stored the program sections of the computer program which can be read in and executed by a computer unit of the control device, can also be used for the transport. The computer unit can have, for example, one or more cooperating microprocessors or the like for this purpose.

The present technology will be explained in more detail with reference to the figures.

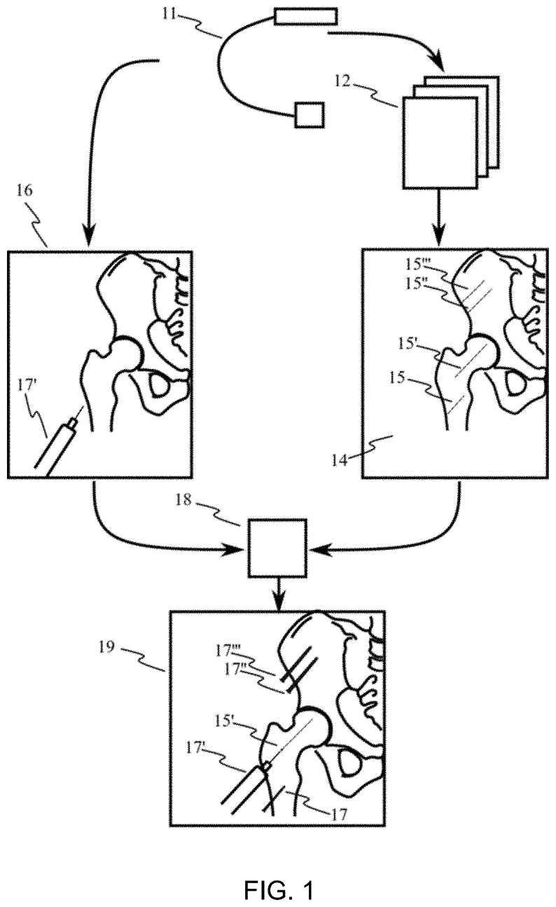

FIG. 1 shows one possible embodiment of a method according to the present technology in which a C-arm X-ray apparatus 11 is used. In preparation for a surgical procedure such as an operation on a hip joint, the C-arm X-ray apparatus 11 can record numerous 2D X-ray images at different recording angles and, using different reconstruction algorithms, generate a 3D image data set 12 and make it available to the method according to the present technology.

The 3D image data set 12 shows the anatomical environment of the hip region. Using the 3D image data set 12, which is displayed on a display device, for example in the form of sectional images in a sectional plane representation 14, the procedure to be carried out is now planned by a user by entering planning trajectories (15, 15′, 15″, 15′″).

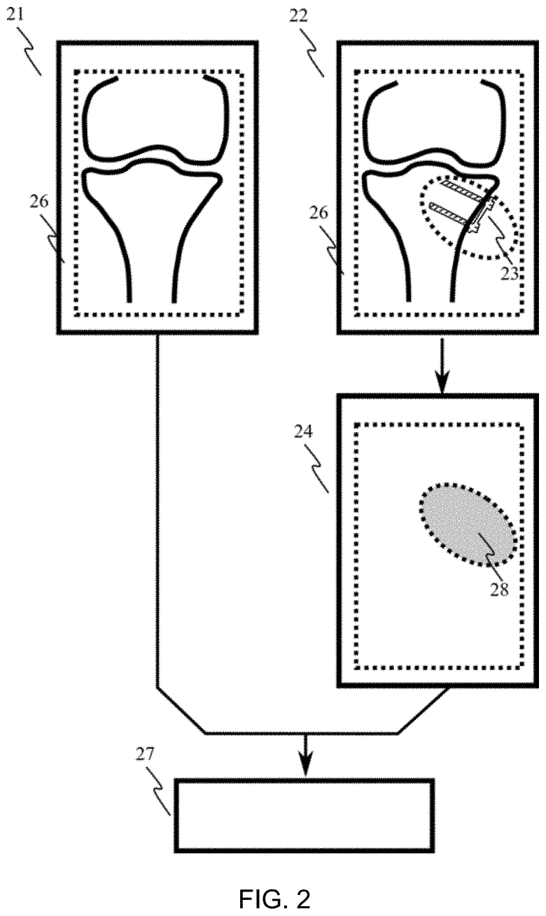

Before or after the input of the planning trajectories (15, 15′, 15″, 15′″), a 2D X-ray image 16, which reproduces the examination region in which the impending procedure is to be carried out, is recorded with the C-arm X-ray apparatus 11. Various foreign objects (17, 17′, 17″, 17′″) can be located in the examination region in a first recording of a 2D X-ray image 16. Thus, for example, clamps and hoses can be located in a recorded examination region which have not yet been inserted into the examination region but are also recorded by the C-arm X-ray apparatus 11 during the recording of the 2D X-ray image 16. A foreign object 17′ in the form of a drill has been introduced into the examination region in FIG. 1. The present technology provides for first examining the recorded 2D X-ray image 16 for foreign objects (17, 17′, 17″, 17′″) and identifying them. If foreign objects (17, 17′, 17″, 17′″) are present, the image regions comprising foreign objects (17, 17′, 17″, 17′″) are masked out for the subsequent determination of an optimum projection geometry 18 between a forward projection from the 3D image data set 12 and the recorded 2D X-ray image 16; an embodiment of the determination of the projection geometry is described in FIG. 2.

After the optimum projection geometry 18 has been determined, the recorded 2D X-ray image 16 can be displayed on the display device, or it can be displayed in addition to the 3D image data set 12, the planning trajectories being displayed correctly in position in the displayed 2D X-ray image by means of the optimum projection geometry.

As the procedure progresses in time, more and more foreign objects (17, 17′, 17″, 17′″) can be located in the examination region, for example screws, hoses, clamps or a drill 17′. If the position of the C-arm X-ray apparatus 11 is unchanged, this increase in foreign objects (17, 17′, 17″, 17′″) in the examination region has no influence on the display of the planning trajectories 15′ in the 2D X-ray image 19. If, for example, the C-arm X-ray apparatus 11 is adjusted or rotated orbitally or angularly and a new 2D X-ray image is recorded, a new optimum projection geometry can be determined in the process, with the foreign objects (17, 17′, 17″, 17′″) located in the examination region being masked out. The planning trajectory (15, 15′, 15″, 15′″) is then displayed in the correct position in the new 2D X-ray image.

FIG. 2 shows an embodiment of the determination of the optimum projection geometry, using the example of a knee joint. In this embodiment, those image areas between the forward projection 21 generated from a 3D image data set can be compared to the image areas of the recorded 2D X-ray image 22 that are not covered by the image regions of the foreign objects 23. According to the present technology, a mask 24 of the 2D X-ray image 22 can be generated, the mask 24 of the 2D X-ray image 22 being generated by masking out the image regions 28 of the foreign object 23.

If no foreign objects are identified in the 2D X-ray image 22, the entire image area may be used as the mask 24; preferably the image edges 26 of the 2D X-ray image recording 22 are also not used for the mask 24. Thus, as shown in FIG. 2 in the case of a knee joint, foreign objects 23 such as screws can be present in a lower leg, which are shown in the 2D X-ray image 22, wherein only the femur and parts of the lower leg not included in the image region 28 of the foreign object 23 are used for determining the projection geometry 27 for the mask 24.

### LIST OF REFERENCE NUMBERS

11 C-arm X-ray apparatus
12 3D image data set
14 Sectional plane representation
15, 15′, 15″, 15′″ Planning trajectory
16, 22 Recorded 2D X-ray image
17, 17′, 17″, 17′″, 23 Foreign object
18, 27 Determination of the projection geometry
19 2D X0ray image with further introduced foreign objects
21 Forward projection
24 Mask 2D X-ray image
26 Image borders
28 Hidden image area of a foreign object

What is claimed is:

1. A method for monitoring images by means of an X-ray apparatus during a surgical procedure by means of 3D-2D registration using at least one foreign object in an examination region, the method comprising:
providing a 3D image data set and displaying at least one layer generated from the 3D image data set on a display device;
inputting a planning trajectory into at least one generated layer of the 3D image data set;
recording a 2D X-ray image of an examination region by means of the X-ray apparatus, wherein the examination region contains the at least one foreign object;
identifying the at least one foreign object in the 2D X-ray image that is not contained in the 3D image data set;
determining an optimum projection geometry using a measure of similarity between the 3D image data set and the 2D X-ray image, wherein the at least one identified foreign object is masked; and
displaying the planning trajectory in the 2D X-ray image on the display device by using the optimum projection geometry.

2. The method of claim 1, wherein the 2D X-ray image is a live image X-ray image recording.

3. The method of claim 1, wherein the determination of the optimum projection geometry takes place by using an iterative and/or parallel optimization method.

4. The method of claim 1, wherein the projection geometry is determined on a fixed grid by using a parallel method on a multiprocessor architecture.

5. The method of claim 1, wherein the optimum projection geometry must satisfy a configurable threshold value of the similarity measure.

6. The method of claim 1, wherein a subset of available geometric degrees of freedom is used to determine the projection geometry.

7. The method of claim 1, wherein the planning trajectory is represented in a second display plane different from a first display plane used to display the at least one layer, and wherein an intersection point of the planning trajectory is displayed in a third display plane.

8. The method of claim 1, wherein, when a plurality of planning trajectories are represented, they are identified differently from one another and/or individual planning trajectories are masked off.

9. The method of claim 1, wherein movements of the X-ray apparatus and/or of an operating table are detected and included in the determination of the optimum projection geometry.

10. The method of claim 1, wherein positions to be approached which facilitate an assessment of an intermediate operation result are determined by a criterion based on the planning trajectories.

11. The method of claim 1, further comprising, before recording the 2D X-ray image, calculating a virtual forward projection from the 3D image data set.

12. The method of claim 11, further comprising, after successful determination of an optimum projection geometry, superimposing the forward projection of the 3D image data set with the 2D X-ray image.

13. The method of claim 1, wherein a new determination of the optimum projection geometry is triggered by operating a hand or foot switch, by changing an X-ray geometry, or by comparing a live image recording to the 2D X-ray image, wherein a new determination is triggered in the event of an excessive difference.

14. The method of claim 1, wherein the 2D X-ray image is recorded before the input of the planning trajectory and/or registration is determined before the input of the planning trajectory.

15. The method of claim 1, wherein the display of the planning trajectory is no longer updated, or is hidden, if no projection geometry is generated which changes or improves the similarity value the previous projection geometry by a fixed relative or absolute value.

16. A device for recording image data sets of X-ray images, in particular a C-arm X-ray apparatus, configured to carry out the method of claim 1, the device comprising:
a memory unit in which a recorded 3D image data set of X-rays is stored;
a reconstruction unit in which the 3D image data set is reconstructed from X-rays to form a 3D volume;
a control unit, said control unit being configured to permit determination of an optimum projection geometry between a forward projection of the 3D image data set and a recorded 2D X-ray image;
an image processing unit for generating a 3D view of the 3D X-ray image data set having variable 3D views and for defining sectional planes for sectional plane image representations; and
a GUI having an image output unit and an input unit for the image processing unit for inputting and changing the sectional planes and planning trajectories.

17. A computer program product having a computer program which can be loaded directly into a memory unit of a control unit for a conical beam computer tomograph, in particular a C-arm X-ray device, with program sections that cause the conical beam computer tomograph to perform the method according to claim 1 when the computer program is executed in the control unit of the conical beam computer tomograph.

18. A computer-readable medium having stored thereon program sections which can be read in and executed by a computer unit in order to perform the method according to claim 1 when the program sections are executed by the computer unit.
